(19) United States
(12) Patent Application Publication
(10) Pub. No.: US 2015/0342767 A1
(43) Pub. Date: Dec. 3, 2015

(54) DEVICE FOR DEPLOYING A COATED STENT IN BLOOD VESSELS

(71) Applicant: Zaza Alexandrovich KAVTELADZE, Moscow (RU)

(72) Inventor: Zaza Alexandrovich KAVTELADZE, Moscow (RU)

(21) Appl. No.: 14/436,742

(22) PCT Filed: Oct. 2, 2013
(86) PCT No.: PCT/US2013/000871

§ 371 Date: Apr. 17, 2015

(30) Foreign Application Priority Data
Oct. 18, 2012 (RU) 2012144360

(51) Int. Cl. A61F 2/95

(52) U.S. Cl. CPC A61F 2/95; A61F 2002/9534

(57) ABSTRACT

The invention relates to medicine, in particular, to vascular and endovascular surgery. The objective of the invention is to develop a device for positioning of a coated stent in blood vessels without a risk of penetration of plaque and blood clot particles inside the stent as well as without a risk of occlusion of lateral branches. The device is made in the form of a frame with an interior coating, the frame being made in the form of two rings interconnected by rods. The frame and rings can have different shapes. The interior coating can be made of a resorbable material with apertures having a size of not more than 100 pm.

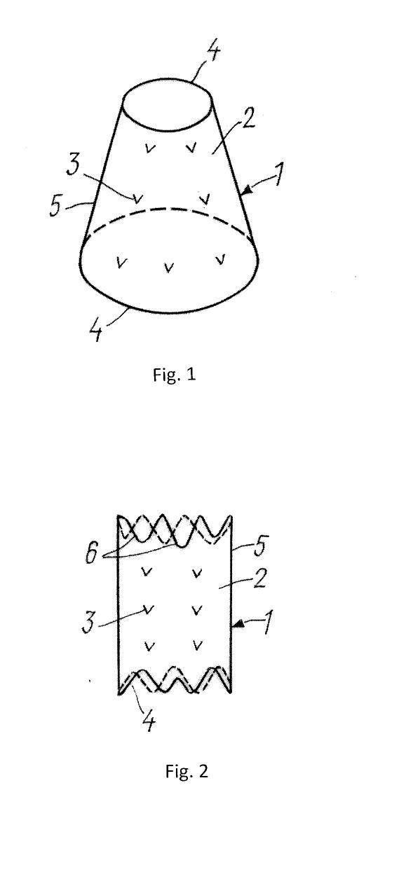

The invention relates to medicine, in particular, to vascular and endovascular surgery.

A blood vessel-implantable stent is known (U.S. Pat. No. 380831, 1997, D24/155). A disadvantage of this stent is in a possibility of penetration of atherosclerotic plaques and blood clots through the stent meshes, their subsequent detachment and entering blood vessels of the hart, brain, lower limbs and other organs, which affects the efficacy of the surgery.

A coated stent-graft for positioning in blood vessels is known. The coating prevents atherosclerotic plaques and blood clots from penetration through the stent meshes. However, a disadvantage of the known stent-graft is in impossibility of using thereof in vessels with lateral branches due to a risk of their occlusion (U.S. Pat. No. 5,628,788, A61F 2/06, 1997).

The objective of the invention is to eliminate the above-indicated disadvantages and to develop a device providing positioning of a coated stent in blood vessels without a risk of penetration of plaque and blood clot particles inside the stent, as well as without a risk of the occlusion of lateral branches.

The objective is achieved by providing a device for positioning a stent in blood vessels, made of a frame with an interior coating, the frame being made in the form of at least two rings interconnected by at least two rods, wherein the device can have a conic or cylindrical shape. The rings of the frame can have a zigzag or U-like shape with uniform or nonuniform arms. The coating can be made of a resorbable material with apertures having a size of not more than 100 μm to prevent atherosclerotic plaques and blood clots from penetration inside the stent, while maintaining blood flow to lateral branches.

The figure shows a general view of the claimed invention.

The device for positioning of a coated stent in the carotid artery, according to the invention, is made of frame 1 having interior coating 2 with apertures 3, the frame being made in the form of at least two rings 4 interconnected by at least two rods 5. Rings 4 have a zigzag or U-like shape with uniform or nonuniform arms 6.

The device is used as follows:

Before positioning a coated stent, frame 1 with interior coating 2 and apertures 3 is introduced into a blood vessel. Rings 4 optimally self-lock to the vessel walls when pushed out from a delivery system. Selection of the shape (conic or cylindrical) of frame 1 depends on the pre-diagnostically determined anatomy of a blood vessel. The zigzag or U-like shape of the rings with uniform or nonuniform arms 6 makes it possible to use frame 1 depending on the anatomy of a blood vessel. Then, a stent (not shown) is disposed inside frame 1. Coating 2 made of a resorbable material (which resorption occurs for a different period of time, depending on material) allows avoiding the development of pressure ulcers inside the lumen of vessels, and apertures 3 with a size of not more than 100 μm prevents atherosclerotic plaques and blood clots from penetration inside the stent, while maintaining blood flow to lateral branches.

Thus, positioning of a stent inside frame 1 having coating with apertures 3 provides double isolation preventing atherosclerotic plaques and blood clots from penetration inside a vessel, while maintaining blood flow to lateral branches, thus increasing the efficacy and safety of stenting.

1. A device for positioning of a coated stent in a blood vessel, characterized in that the device is made of a frame with an interior coating, the frame being made in the form of at least two rings interconnected by at least two rods.

2. The device of claim 1, characterized in that the frame may have a conical or cylindrical shape.

3. The device of claim 1, characterized in that the rings have a zigzag or U-like shape with uniform or nonuniform arms.

4. The device of claim 1, characterized in that the interior coating can be made of a resorbable material with apertures having a size of not more than 100 μm.
